(19) United States
(12) Patent Application Publication
(10) Pub. No.: US 2025/0114302 A1
(43) Pub. Date: Apr. 10, 2025

(54) Functionalized Hydrogels for Tissue Repair

(71) Applicants: The Trustees of the University of Pennsylvania, Philadelphia, PA (US); The United States Government As Represented By The Department Of Veterans Affairs, Washington, DC (US)

(72) Inventors: Emily Sharp, Philadelphia, PA (US); Robert Leon Mauck, Philadelphia, PA (US); Sarah Elizabeth Gullbrand, Flourtown, PA (US); Jason Alan Burdick, Philadelphia, PA (US); Zhiliang Cheng, Newtown, PA (US); Karen Xu, Philadelphia, PA (US)

(21) Appl. No.: 18/904,336

(22) Filed: Oct. 2, 2024

Related U.S. Application Data
(60) Provisional application No. 63/588,047, filed on Oct. 5, 2023.

(51) Int. Cl. A61K 9/06; A61K 9/00; A61K 47/36

(52) U.S. Cl. CPC A61K 9/06; A61K 9/0024; A61K 47/36

(57) ABSTRACT

A composition, comprising: a hydrogel comprising crosslinked polymer chains, at least some of the crosslinked polymer chains having an agent coupled thereto. A method, comprising curing a precursor composition so as to give rise to a composition according to the present disclosure. A precursor composition, comprising: polymer chains bearing pendant groups; a crosslinker adapted to couple pendant groups to one another; and a functionalizer adapted to couple to a pendant group. A method, comprising curing a precursor composition according to the present disclosure so as to give rise to a composition, the composition optionally being a composition according to the present disclosure. A method, comprising: forming a hydrogel; and coupling the hydrogel to an agent, the coupling being effected by coupling (1) a pendant group borne by a polymer chain of the hydrogel and (2) a coupler associated with the agent.

### CROSS-REFERENCE TO RELATED APPLICATIONS

This application claims benefit to U.S. Provisional Application No. 63/588,047, “Functionalized Hydrogels For Tissue Repair,” filed Oct. 5, 2023. All foregoing applications are incorporated by reference in their entireties for any and all purposes.

### GOVERNMENT RIGHTS

This invention was made with government support under AR056624 and AR079875 awarded by the National Institutes of Health. The government has certain rights in the invention.

### TECHNICAL FIELD

The present disclosure relates to the field of biomaterials.

### BACKGROUND

Numerous hydrogel biomaterials have been developed for the repair of connective tissues; however, a very limited number of multifunctional hydrogels have been developed that combine wound repair with drug delivery. Existing approaches, however, are restricted in the level of controlled release of the therapeutic they can achieve, risk inefficiency and side effects due to physiological clearance of the therapeutic, and can also be restricted to a specific tissue type and function. Accordingly, there is a long-felt need in the art for improved biomaterials, particularly for such materials useful in repair of connective tissues.

### SUMMARY

In meeting the described long-felt needs, the present disclosure provides a composition, comprising: a hydrogel comprising crosslinked polymer chains, at least some of the crosslinked polymer chains having an agent coupled thereto.

Also provided is a method, comprising curing a precursor composition so as to give rise to a composition according to the present disclosure.

Further provided is a precursor composition, comprising: polymer chains bearing pendant groups; a crosslinker adapted to couple pendant groups to one another; and a functionalizer adapted to couple to a pendant group.

Additionally disclosed is a method, comprising curing a precursor composition according to the present disclosure so as to give rise to a composition, the composition optionally being a composition according to the present disclosure.

Further provided is a method, comprising: forming a hydrogel; and coupling the hydrogel to an agent, the coupling being effected by coupling (1) a pendant group borne by a polymer chain of the hydrogel and (2) a coupler associated with the agent.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIGS. 1A-1C: FIG. 1A) Non-modified (NorHA) and azide-modified (NorHA-TPA) hydrogels incubated in PBS or 30 μM AF-488-DBCO. FIG. 1B) Fluorophore intensity of NorHA and NorHA-TPA hydrogels when incubated in varying concentrations of AF-488-DBCO. FIG. 1C) NorHA-TPA 1 week and 1 month post-incubation in 30 μM AF-488-DBCO.

FIGS. 2A-2B: FIG. 2A) Schematic of sPLA2i-loaded micelles, which were DBCO-modified for this study. FIG. 2B) NorHA and NorHA-TPA hydrogels incubated in 30 μM micelles-DBCO labelled with Rhodamine.

FIG. 3: Cell viability and proliferation for controls (no hydrogel, AF cells only, black dots) and with NorHA-TPA exposure (green dots). An increase in (negative) absorbance corresponds to an increase in cells number.

FIGS. 4A-4B: FIG. 4A) Hydrogels with varying levels of TPA incubated in AF-488-DBCO and washed for up to 4 weeks. FIG. 4B) Fluorescent intensity when varying TPA modification. n=4.

FIGS. 5A-5D: FIG. 5A) Equilibrium moduli for NorHA and NorHA-TPA4 hydrogels of varying weight percent. FIG. 5B) Gelation kinetics of hydrogels with respective Tau values. FIG. 5C) Day 3 images of AF cells seeded onto hydrogels. FIG. 5D) AlamarBlue data of cells seeded onto hydrogels for up to a week. n=>3.

FIG. 6: AF repair via NorHA-TPA (blue) before and after physiologic loading for 10,000 cycles. n=1.

FIG. 7: Example strategy for azide functionalization of norbornene-modified hyaluronic acid (NorHA) using thiol-PEG-azide (TPA). DBCO-modified nanocarriers can bind to azide-modified hydrogels via click chemistry.

FIGS. 8A-8C: FIG. 8A) Complex viscosity as a function of shear rate at 1% strain for NorHA-TPA4 hydrogel precursor solutions. There was no significant difference between these solutions and NorHA counterparts (not shown). FIG. 8B) Storage modulus (G′) of precursor solutions when exposed to 10 mW/cm² UV light at t=0 until t=180. The observed differences in G′ between weight percentages are statistically significant. Shown are the average curves for n=2-6 per group without the error bars for clarity. FIG. 8C) Equilibrium modulus when varying NorHA weight percentage for 50% crosslinked NorHA and NorHA-TPA4 hydrogels.

FIGS. 9A-9B: FIG. 9A) AlamarBlue results normalized to d1 measurements for each group. FIG. 9B) Actin/DAPI stain of bovine AF cells seeded onto either NorHA (left) or NorHA-TPA (right) hydrogels at d3 post-seeding and 20× magnification.

FIGS. 10A-10B: FIG. 10A) Images of various hydrogels incubated in AF-488-DBCO for 1 hour and washed in PBS for up to 4 weeks. FIG. 10B) Quantification of fluorescent intensity of hydrogels when varying thiol-PEG-azide (TPA) functionalization. n=4 hydrogels/group.

FIG. 11: A cruciate-puncture injury was created at the center of the disc and was either repaired with a 5 wt %, 80% crosslinked NorHA-TPA4 hydrogel (Blue) or left unrepaired (Green). The explant was cycled 10,000 times under physiological loading.

FIGS. 12A-12B. Mechanical characterization of hydrogels when varying FIG. 12A) weight percent and FIG. 12B) TS functionalization in 3 wt % hydrogels. n>4 hydrogels/group.

FIGS. 13A-13C. FIG. 13A) Biotin-Cy5 attachment to hydrogels with varying levels of TS functionalization (Scale=2 mm) and their FIG. 13B) normalized fluorescent intensity (with SD) and FIG. 13C) percent loss of fluorescence (with SEM). n>3 hydrogels/group.

FIGS. 14A-14B: FIG. 14A) Changes in hydrogel mechanics and FIG. 14B) normalized fluorescent intensity with implantation time. n>3 hydrogels/group.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “about” and “substantially,” may not be limited to the precise value specified, in some cases. In at least some instances, the approximating language can correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” can refer to plus or minus 10% of the indicated number. For example, “about 10%” can indicate a range of 9% to 11%, and “about 1” can mean from 0.9-1.1. Other meanings of “about” can be apparent from the context, such as rounding off, so, for example “about 1” can also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition made of A and B only. Any documents cited herein are incorporated by reference in their entireties for any and all purposes.

Any embodiment or aspect provided herein is illustrative only and does not limit the scope of the present disclosure or the appended claims. Any part or parts of any one or more embodiments or aspects can be combined with any part or parts of any one or more other embodiments or aspects.

Although the disclosed technology is illustrated here by reference to NorHA-based hydrogels used in the context of annulus fibrosis repaid, it should be understood that these illustrative embodiments are exemplary only and do not limit the scope of the disclosed technology or the appended claims.

The intervertebral discs (IVDs) are the soft tissues bridging adjacent spinal vertebrae, providing flexibility, load transfer, and shock absorption during multi-axial movement. IVDs are comprised of the highly organized annulus fibrosus (AF) that surrounds the gel-like nucleus pulposus (NP), which are separated from adjacent vertebrae by thin cartilaginous end-plates (CEPs). Trauma, overuse, or degeneration can result in AF tears or ruptures that disrupt the crucial role that the IVD plays in daily activities and can lead to NP herniation. The standard of care after herniation is microdiscectomy, where the herniated NP tissue is surgically excised. While effective in relieving symptoms that arise from herniation (i.e., pain, motor and sensory deficits), the mechanical integrity of the AF remains compromised, and a large defect persists through which remaining NP tissue can reherniate.

Successful annular repair following herniation offers immense potential in transforming clinical practice and creating superior surgical alternatives and outcomes for patients. To that end, next-generation multifunctional biomaterials enable complete customization to achieve ideal design criteria. In addition to common design criteria like biocompatibility and biodegradability, an ideal annular repair material would be injectable and have the capacity for controlled delivery of a therapeutic. Injectability enables the repair material to interdigitate with the AF to precisely fill the complex defect created by AF tears or ruptures. Controlled delivery of a therapeutic would not only minimize patient discomfort (by controlling inflammation, for example), but can also create a more favorable environment for tissue regeneration and annular repair. However, many proposed strategies for annular repair, such as hyaluronic acid and fibrin-based materials, lack this significant controlled delivery component.

Here, we developed an injectable hydrogel capable of spatiotemporal controlled delivery via click chemistry. Click chemistry describes a class of chemical reactions where two small molecules rapidly and strongly bind to one another. Specifically, we utilize strain-promoted azide-alkyne cycloaddition (SPAAC) click reactions. Norbornene-modified hyaluronic acid (NorHA) hydrogels were azide-modified to click- functionalize the hydrogels, which was first confirmed using alkyne-modified fluorophores. Towards enabling controlled delivery, we demonstrate that alkyne-modified micelles can selectively attach to these azide-modified NorHA hydrogels. It was confirmed that this azide-modification is cytocompatible with AF cells in vitro. While additional characterization and customization of these hydrogels is necessary for optimal use as an annular repair material, this novel multifunctional biomaterial has the potential to revolutionize how we approach dense connective tissue repair and regeneration.

### Methods

Hydrogel Synthesis and Click-Functionalization: NorHA synthesis was accomplished using established protocols. UV photocrosslinking of NorHA hydrogels was achieved using a di-thiol photocrosslinker that reacts with a portion of the pendant norbornene groups via a thiol-ene Michael addition. To click-functionalize these hydrogels, a thiol-PEG-azide (TPA) (MW: 1 kDa) was added to the hydrogel solution in a 50:50 ratio with the photocrosslinker (i.e., 50% of available norbornene groups were azide-modified and the remaining 50% were used to crosslink the hydrogel). It should be understood that this is a variable that can be adjusted to achieve a desired delivery profile, as it can be varied depending on the user's preferences and also depending on the particular application of interest. In some of the data provided herein, the majority of the hydrogels still have 50% of the norbornenes crosslinked but only about 10-15% of the remaining norbornenes functionalized with azide. The exact value of functionalization is represented by the number after TPA; for example, TPA1=3.3% functionalized; TPA2=6.6% functionalized; TPA3=9.9% functionalized, and TPA4=13.2% functionalized. The pendant groups of an exemplary hydrogel can be crosslinked to a degree of from 1% to about 50%, 60%, 70%, 80%, 90% or even greater, depending on the particular application. The degree of functionalization can be, for example, from about 0.5% up to about 50%, up to about 45%, up to about 40%, up to about 35%, up to about 30%, up to about 25%, up to about 20%, up to about 15%, or even up to about 10%. A proportion of pendant groups can be non-crosslinked and non-functionalized; such a proportion can be, for example, up to about 50%, up to about 45%, up to about 40%, up to about 35%, up to about 30%, up to about 25%, up to about 20%, up to about 15%, up to about 10%, or even up to about 5%.

Click Validation: Confirmation of click-functionalization was achieved by incubating non-modified (NorHA) and azide-modified (NorHA-TPA) hydrogels in an alkyne-modified fluorophore solution (30 μM Alexa Fluorophore (AF)-488-Dibenzocyclooctyne (DBCO)) for 1 hour and assessing attachment via confocal microscopy. These same hydrogels were imaged after 1 week and 1 month of incubation in a PBS bath. The concentration of fluorophore was also varied to demonstrate concentration-dependent attachment via the click reaction. Quantification of the fluorophore attachment was carried out in ImageJ.

Micelle Attachment: DBCO-modified micelles labelled with Rhodamine were fabricated. As a proof of concept, these micelles were loaded with a secretory phospholipase A2 inhibitor (sPLA2i). sPLA2 mediates inflammation and tissue damage and is upregulated in injured AF scenarios. Non-modified (NorHA) and azide-modified (NorHA-TPA) hydrogels were incubated in a 30 μM solution of these micelles overnight and then were vigorously shaken (500 RPM) for 24 hours prior to imaging via confocal microscopy.

Cytotoxicity Evaluation: To ensure azide-modification did not impact hydrogel biocompatibility, we performed a non-contact cytotoxicity assay using bovine AF cells (P3-P4). AF cells were seeded in 96-well plates at a seeding density of 6,000 cells/cm². Cells were incubated at 37° C. in basal media for 24 hours before exposure. Cells either received no exposure or were indirectly exposed to NorHA-TPA hydrogels using 0.8 μm transwell inserts (n=12/treatment group). AlamarBlue was used to assess cell viability and proliferation by measuring absorbance (at 590 nm) at days 1, 3, and 7 of exposure. Measurements were subtracted from media only (i.e., no cells) controls and plotted using GraphPad (Statistics: unpaired t-tests).

NorHA hydrogels are useful for biomedical applications due to their injectability and inherent biocompatibility and biodegradability. However, these hydrogels lack a mechanism for controlled therapeutic delivery, which is desirable in nearly all biomedical applications. For instance, an ideal AF repair material would possess all these characteristics to precisely repair the wound defect while utilizing controlled delivery to minimize inflammation and promote tissue regeneration.

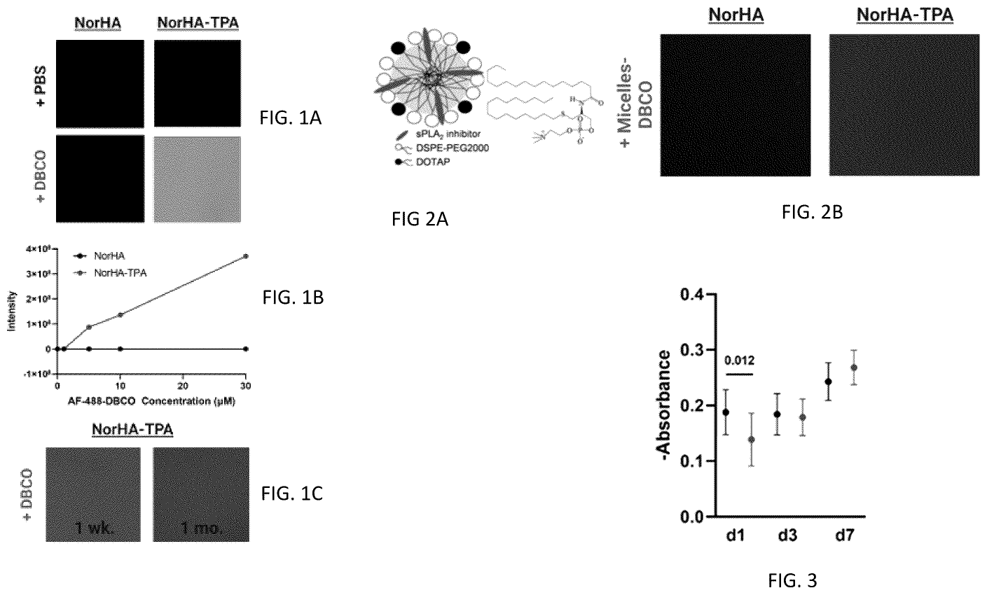

Here, we showed that the incorporation of thiol-PEG-azide (TPA) successfully click-functionalizes NorHA hydrogels. This was demonstrated through the selective attachment of DBCO-modified fluorophores to NorHA-TPA (FIG. 1A), which was stable for at least one month. This shows that therapeutic-loaded nanoparticles can be attached to NorHA-TPA and subsequently localized to the site of injury for delivery. We demonstrated this with loaded DBCO-modified micelles (FIG. 2B). Notably, both the therapeutic and the specific type of nanoparticles (e.g., micelles, liposomes, polymeric, etc.) can be altered based on the specific delivery requirement for the hydrogel system. Additionally, the number of particles attached can be tailored by varying solution concentration (FIG. 1B) or by altering the amount of TPA. Regardless, the attachment of DBCO-modified particles is highly stable for considerable durations (FIG. 1C). Importantly, TPA incorporation did not impact AF cell viability or proliferation (FIG. 3).

### Summary-NorHA-TPA Hydrogels

NorHA-TPA hydrogels are novel multifunctional biomaterials with immense potential to revolutionize how we approach dense connective tissue repair and regeneration, especially within the field of intervertebral disc repair following herniation.

Here, we have shown that NorHA-TPA hydrogels are (1) ‘clickable,’ and that the degree of click-functionalization can be customized; (2) non-cytotoxic; (3) injectable (i.e., shear-thinning); (4) rapidly gelled upon UV exposure; (5) able to be tailored to meet specific mechanical property criteria (via weight percentage); and (6) resident after 10,000 cycles under physiologic loading ex vivo.

It should be understood, however, that the disclosed technology is not limited to NorHA hydrogels, as other hydrogels can be used. Example hydrogels include polyvinyl alcohol (PVA) hydrogels; polyethylene glycol (PEG) hydrogels; sodium polyacrylate hydrogels; polyacrylamide hydrogels; poly(n-isopropylacrylamide) (PNIPAM) hydrogels; cellulose hydrogels; chitosan hydrogels; alginate hydrogels; gelatin hydrogels; hyaluronic acid hydrogels; fibrin hydrogels; collagen hydrogels; dextran hydrogels; cellulose hydrogels; starch hydrogels; poly(2-hydroxyethyl methacrylate) (PHEMA) hydrogels; polyvinylpyrrolidone (PVP) hydrogels; poly(ethylene oxide) (PEO) hydrogels; poly(lactic-co-glycolic acid) (PLGA) hydrogels; polyurethane hydrogels; poly(ethyleneimine) (PEI) hydrogels, and others. Biocompatible hydrogels are considered especially suitable.

### Additional Results

Hydrogel Fabrication: NorHA synthesis and hydrogel fabrication were performed. Percent crosslinking was 50% (unless otherwise specified) and hydrogel weight percent varied between 3-5%. Click-functionalization was achieved via the addition of thiol-PEG-azide (TPA) to the hydrogel precursor solution. The level of TPA modification varied from ˜3.3% (TPA1) to ˜13.2% (TPA4). Upon UV exposure, hydrogel photocrosslinking and azide-modification occur simultaneously.

Click Validation: Here, the click counterpart to azide is dibenzocyclooctyne (DBCO). 3 wt % NorHA and NorHA-TPA hydrogels, of varying degrees of azide-modification, were fabricated, allowed to swell overnight in PBS, and incubated in a 30 μM DBCO-modified Alexa Fluorophore (AF-488-DBCO) solution for 1 hour. Hydrogels were imaged via an Axiozoom microscope for up to 4 weeks and incubated in PBS on an orbital shaker at 4 C between imaging.

Mechanical and Rheological Properties: 3 and 5 wt % NorHA and NorHA-TPA4 hydrogels were tested using a stress relaxation protocol (10% strain at 0.05%/s followed by stress relaxation for 10 min.) to determine the equilibrium modulus. To assess gelation kinetics, 5 wt % NorHA and NorHA-TPA4 precursor solutions were exposed to UV light for 180 seconds and storage modulus was assessed via rheology at 1% strain at 1 Hz. Curves were fitted with a one phase association exponential fit to determine Tau values (i.e., time to gelation).

Cytocompatibility: Bovine AF cells (40,000 cells/cm2) were seeded on sterile 3 wt % NorHA and NorHA-TPA4 hydrogels additionally modified with thiol-RGD peptide (1 mM). Cell viability and proliferation were monitored over 7 days via the AlamarBlue assay, and cells were imaged on day 3 to investigate cell adhesion.

Gel Retention in AF defects: A cruciate injury to the AF was created in bovine caudal discs, and the defect was either left empty or filled with 5 wt % NorHA-TPA4 with a crosslinking density of 80%. The explant was cycled under physiologic loading (300 N) for 10,000 cycles using an Instron.

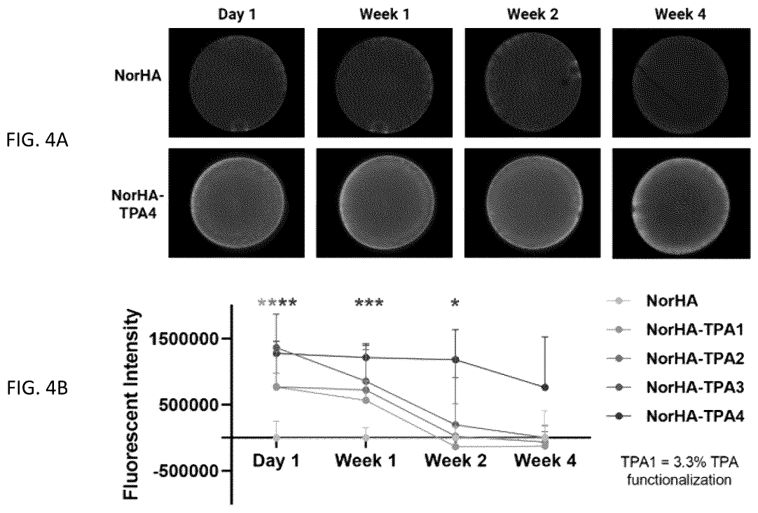

FIG. 4 illustrates FIG. 4A) fluorescence results for hydrogels with varying levels of TPA incubated in AF-488-DBCO and washed for up to 4 weeks. FIG. 4B) Fluorescent intensity when varying TPA modification. n=4.

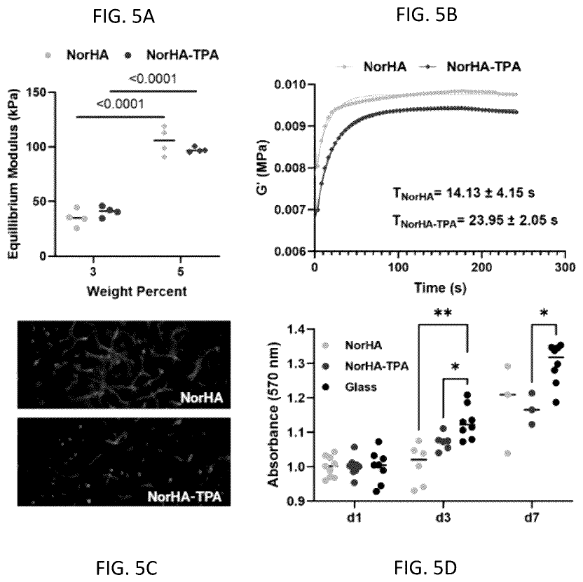

FIG. 5: FIG. 5A) Equilibrium moduli for NorHA and NorHA-TPA4 hydrogels of varying weight percent. FIG. 5B) Gelation kinetics of hydrogels with respective Tau values. FIG. 5C) Day 3 images of AF cells seeded onto hydrogels. FIG. 5D) AlamarBlue data of cells seeded onto hydrogels for up to a week. n=>3.

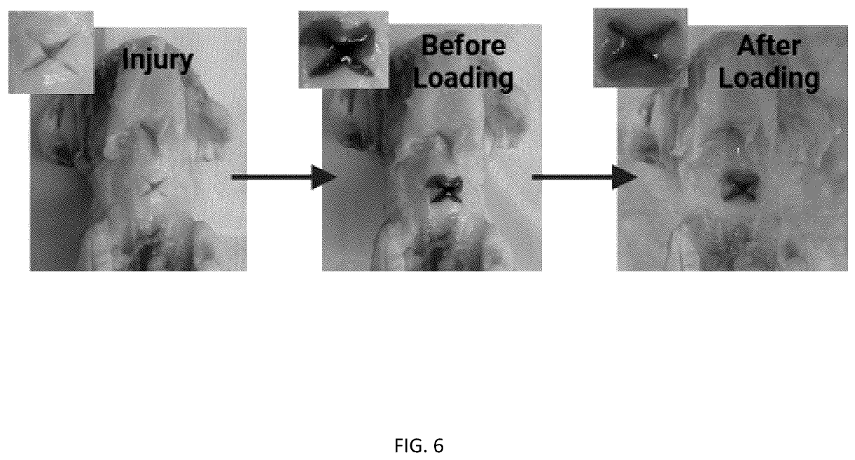

FIG. 6: AF repair via NorHA-TPA (blue) before and after physiologic loading for 10,000 cycles. n=1.

As shown, DBCO-modified fluorophores preferentially bound to NorHA-TPA hydrogels, and the degree of this attachment can be tailored based on the degree of azide-modification (FIG. 4). Specifically, increasing azide-modification increased DBCO-fluorophore attachment and prolonged length of attachment. Hydrogel mechanics can be tailored, for example, by changing weight percent (FIG. 5A). While the addition of TPA did not impact bulk hydrogel mechanics, it slightly slowed gelation kinetics as indicated by larger Tau values for NorHA-TPA hydrogels; overall, gelation remained rapid (<30 s) (FIG. 5B). Additionally, AF cells readily adhered to the hydrogels by day 3 (FIG. 5C) and cell proliferation increased over time (FIG. 5D). Lastly, NorHA-TPA gel mediated repair of an annular defect and remained within the defect following 10,000 physiological compression cycles (FIG. 6).

NorHA hydrogels have particular application to tissue repair due to their injectability, biocompatibility, and biodegradability. As shown, NorHA hydrogels can be functionalized to enable controlled delivery via secondary azide-alkyne click reactions. The addition of TPA enhanced attachment of DBCO-modified fluorophores and varying the degree of azide-modification enabled tailoring of the extent and duration of attachment. Thus, DBCO-modified therapeutics or nanocarriers can be effectively tethered to the hydrogel, localizing the therapeutic with considerable control over its release profile. Further, TPA incorporation did not adversely impact hydrogel mechanics, mechanical tunability, or gelation kinetics. Cell adhesion and proliferation were also not affected by TPA addition. Ex vivo testing demonstrated that NorHA-TPA remained within the annular defect over 10,000 cycles of physiologic loading.

As shown, the disclosed hydrogel has application to repairing the AF following herniation, transforming clinical practices and creating superior surgical alternatives and outcomes for patients. This novel multifunctional biomaterial can also be employed to repair multiple different tissue types and injuries.

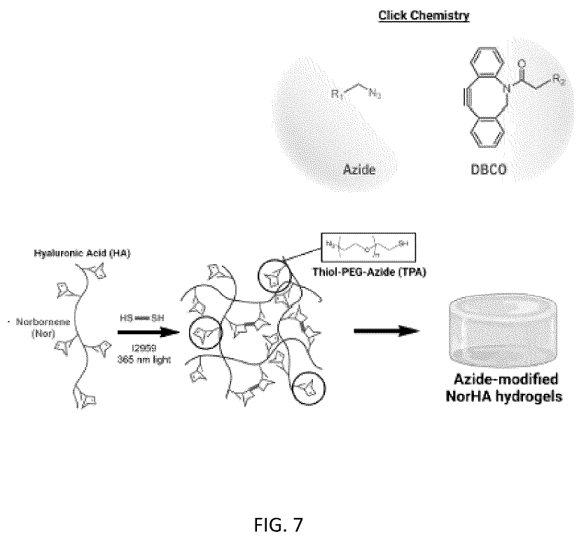

FIG. 7 provides a non-limiting, example strategy for azide functionalization of norbornene-modified hyaluronic acid (NorHA) using thiol-PEG-azide (TPA). DBCO-modified nanocarriers can bind to azide-modified hydrogels via click chemistry.

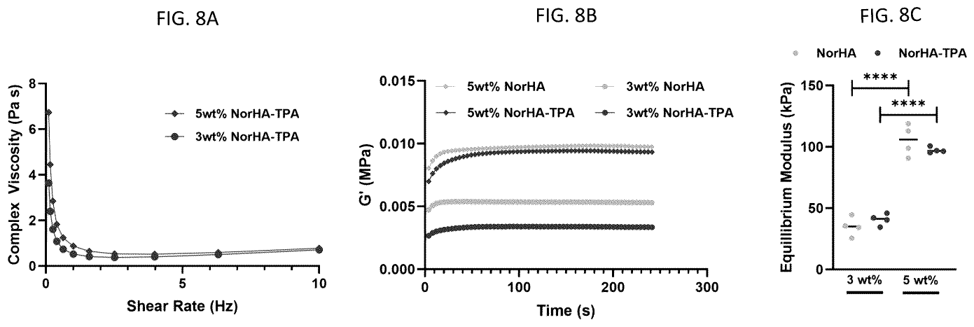

FIGS. 8A-8C provide further exemplary results. FIG. 8A provides complex viscosity as a function of shear rate at 1% strain for exemplary NorHA-TPA4 hydrogel precursor solutions. There was no significant difference between these solutions and NorHA counterparts (not shown). FIG. 8B provides storage modulus (G′) of precursor solutions when exposed to 10 mW/cm2 UV light at t=0 until t=180. The observed differences in G′ between weight percentages are statistically significant. Shown are the average curves for n=2-6 per group without the error bars for clarity. FIG. 8C provides equilibrium modulus when varying NorHA weight percentage for 50% crosslinked NorHA and NorHA-TPA4 hydrogels.

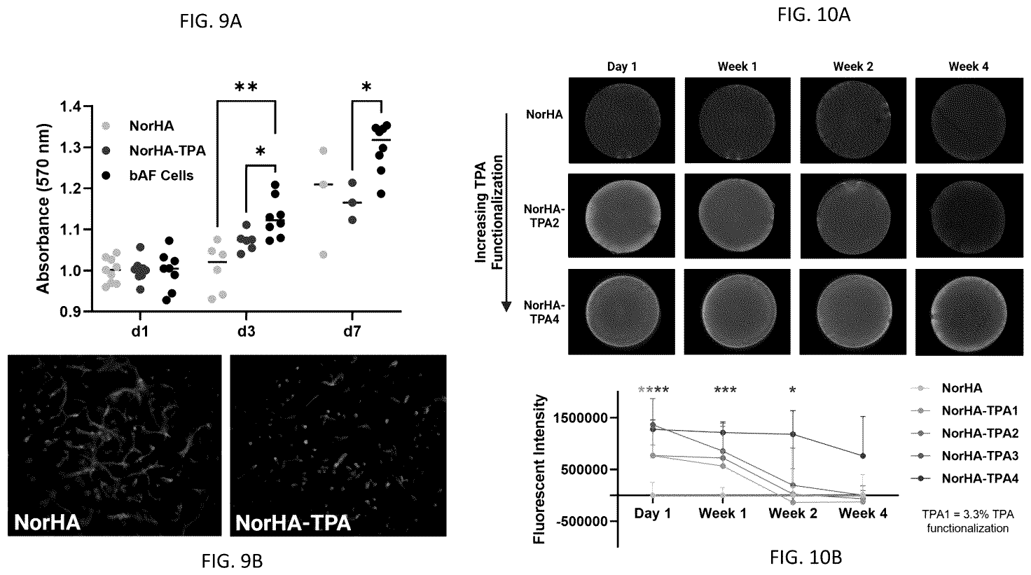

FIGS. 9A-9B provide further results. FIG. 9A provides AlamarBlue results normalized to d1 measurements for each group. FIG. 9B provides an actin/DAPI stain of bovine AF cells seeded onto either NorHA (left) or NorHA-TPA (right) hydrogels at d3 post-seeding and 20× magnification.

FIGS. 10A-10B provide additional results. FIG. 10A provides images of various hydrogels incubated in AF-488-DBCO for 1 hour and washed in PBS for up to 4 weeks. FIG. 10B provides a quantification of fluorescent intensity of hydrogels when varying thiol-PEG-azide (TPA) functionalization. n=4 hydrogels/group.

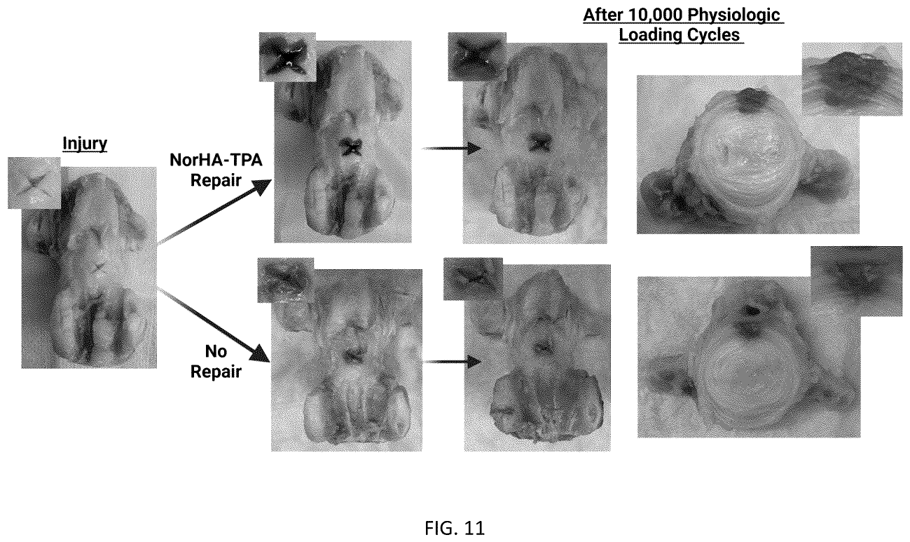

FIG. 11 illustrates a cruciate-puncture injury created at the center of the disc and was either repaired with a 5 wt %, 80% crosslinked NorHA-TPA4 hydrogel (Blue) or left unrepaired (Green). The explant was cycled 10,000 times under physiological loading. As shown, the hydrogel persisted at the repair site after the loading cycles.

### Further Disclosure

Intervertebral disc (IVD) herniations, where the nucleus pulposus (NP) extrudes beyond the boundaries of the annulus fibrosus (AF), disrupts IVD mechanical function and results in significant pain for patients. The gold standard of care, microdiscectomy, is effective in relieving the immediate symptoms of herniation, however, long-term outcomes can be poor, with patients experiencing as much, if not more, pain at one-year compared to those who did not undergo microdiscectomy. Moreover, microdiscectomy does not restore the mechanical integrity of the AF, allowing for reherniations to occur through the unrepaired defect, and it does not address the inflammatory cascade that accompanies herniation and complicates endogenous healing. A variety of biomaterial-based strategies for AF repair have been explored in recent years, but generally lack functionality for therapeutic delivery.

As described herein, we disclose a norbornene-modified hyaluronic acid (NorHA) hydrogel that satisfies AF repair criteria (biocompatible, injectable, mechanical compatibility) and is capable of spatiotemporal controlled delivery via secondary reactions between the hydrogel and a desired therapeutic (drug, biologic, nanocarrier, etc.). This delivery system employs biotin-streptavidin chemistry, where the hydrogel is modified with streptavidin and the desired therapeutic is modified with biotin. Here, we evaluated the effect of NorHA functionalization, durability of the biotin-streptavidin reaction with time in vitro, and the behavior of the hydrogel in vivo.

Hydrogel Synthesis and Functionalization: NorHA synthesis and hydrogel fabrication were accomplished using established protocols. Percent crosslinking was 50% and weight percent was 3% (unless otherwise specified). NorHA functionalization was achieved by incorporating thiol-streptavidin (TS) into the precursor solution. Percent functionalization is referred to in units of TS where 1 unit is roughly equivalent to 3.3% functionalization. Upon UV exposure, hydrogel photocrosslinking and streptavidin functionalization occur simultaneously.

Mechanical Characterization: 3 and 5 wt % hydrogels with varying degrees of TS functionalization (3, 6, and 9 TS) were tested using a stress relaxation protocol (10% strain at 0.05%/s followed by stress relaxation for 10 min) to determine equilibrium modulus.

Secondary Reaction Validation: NorHA hydrogels with varying levels of functionalization (0, 3, 6, and 9 TS) were fabricated and allowed to swell overnight prior to being incubated in 30 μM biotin-modified Cy5 fluorophore for 1 hour at room temperature. Hydrogels were then washed in PBS with 10% fetal bovine serum and 2% penicillin-streptomycin at 37° C. overnight before imaging via AxioZoom microscope. Images were taken every 7 days, with media changed every 3-4 days for 4 weeks. Media was collected to measure fluorescence loss with time using a microplate reader. Images were quantified with ImageJ and NorHA fluorescent intensity values were subtracted out to account for non-specific fluorescence attachment at each timepoint.

In vivo Evaluation: NorHA and NorHA-TS4 hydrogels were implanted subcutaneously into Sprague-Dawley rats for 7 and 28 days, monitoring hydrogel mechanical properties and retention of biotin-modified fluorophore to the hydrogels, as detailed previously.

Statistical Analysis: All statistics were conducted in GraphPad Prism with a significance threshold of p<0.05. Outliers were removed from each dataset and significant differences were detected using the appropriate ANOVA test.

### Results

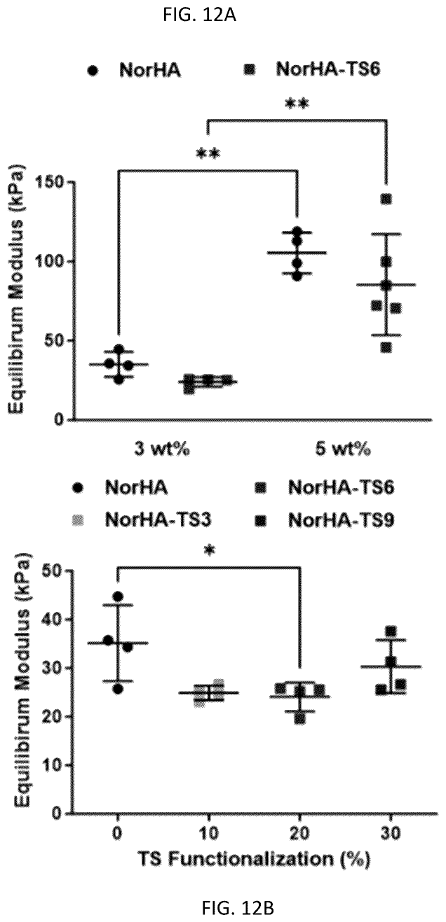

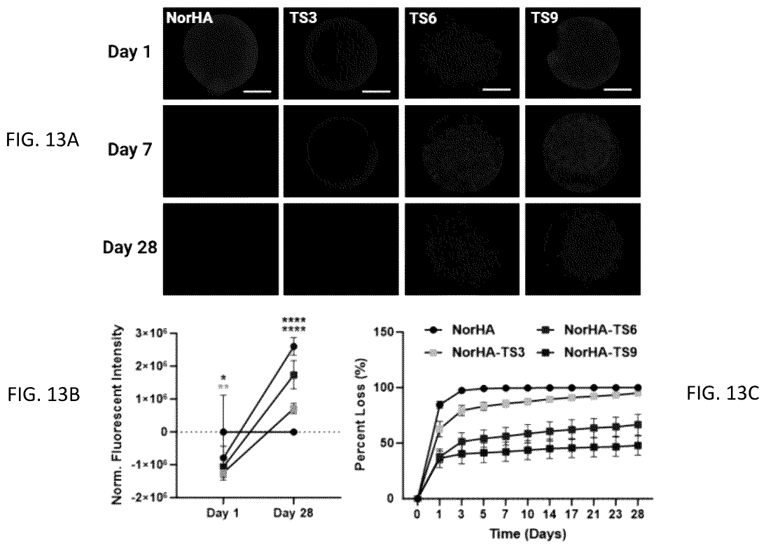

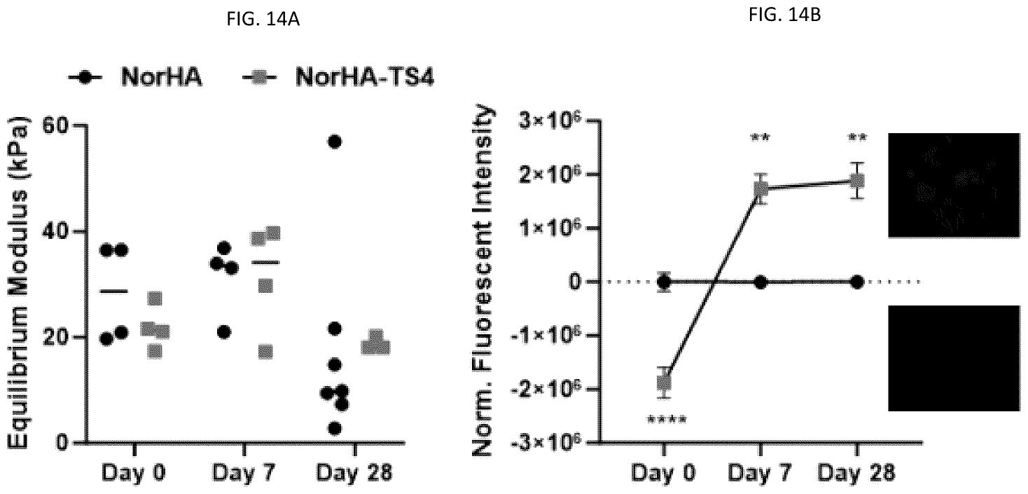

As anticipated, the incorporation of TS did not significantly alter the mechanical properties of the hydrogels while still allowing for mechanical tunability via a change in weight percent (FIG. 12A). Varying the degree of TS functionalization at a constant weight percent did not meaningfully alter the mechanical properties of the hydrogel (FIG. 12B). After incubation in a biotin-modified fluorophore, NorHA hydrogels showed increased fluorescence at Day 1 compared TS hydrogels but lost all fluorescence by Day 7 (FIG. 13A). Hydrogels with TS functionalization had stable attachment of the biotin-modified fluorophore for at least 28 days (FIG. 13A-B). Importantly, varying the degree of TS functionalization resulted in differential release of the biotin-modified fluorophore, with increased TS functionalization resulting in decreased percent loss at each timepoint (FIG. 13C). TS6 and TS9 hydrogels lost significantly less fluorophore into the washing media compared to non-modified controls across all time points, while fluorescence loss from TS3 hydrogels was not statistically significant from that of non-modified hydrogels at any timepoint. In vivo evaluation demonstrated that the mechanical properties of the hydrogels do not significantly change with implantation time, but there was a decreasing trend with time that suggests hydrogel degradation (FIG. 14A). As with in vitro examinations, TS hydrogels maintained their fluorophore attachment for 28 days in vivo compared to NorHA hydrogels (FIG. 14B).

### Discussion

NorHA hydrogels are promising candidates for tissue repair due to their injectability, biocompatibility, and biodegradability; yet are lacking in controlled delivery capacity. Here, we demonstrate that functionalization of NorHA hydrogels with TS does not adversely impact hydrogel mechanics or mechanical tunability. The addition of TS enables a strong, precise secondary reaction between the functionalized hydrogel and a biotin-modified fluorophore that is customizable based on the extent of TS functionalization and is stable both in vitro and in vivo. The secondary reaction can be utilized to localize a therapeutic or delivery carrier to the hydrogel utilizing this same mechanism.

### Significance

The disclosed functionalized hydrogels have utility in repairing the AF or other dense connective tissues through achieving spatiotemporal controlled delivery of a therapeutic to engage endogenous healing capabilities. These materials can thus transform clinical practice, creating superior surgical alternatives and outcomes for patients.

### Aspects

The following Aspects are illustrative only and do not limit the scope of the present disclosure or the appended claims. Any part or parts of any one or more Aspects can be combined with any part or parts of any one or more other Aspects.

Aspect 1. A composition, comprising: a hydrogel comprising crosslinked polymer chains, at least some of the crosslinked polymer chains having an agent coupled thereto.

An agent can be coupled directly to a polymer chain, although this is not a requirement. In some embodiments, an agent can be coupled to a pendant group that is itself coupled to a polymer chain.

As described elsewhere herein, the coupling can be accomplished via, for example, click chemistry and by biotin-streptavidin reaction. Example click chemistry reactions include, as but some examples, copper-catalyzed azide alkyne cycloaddition (CuAAC); strain-promoted azide-alkyne cycloaddition (SPAAC); strain-promoted alkyne-nitrone cycloaddition (SPANC); Diels-Alder reaction; inverse electron demand Diels-Alder reaction; thiol-ene reaction; thiol-yne reaction; thiol-epoxy reaction; thiol-isocyanate reaction; thiol-Michael addition reaction; aza-Michael addition reaction; amino-epoxy ring-opening reaction; sulfur fluorine exchange (SUFEX) reaction; alkene and tetrazine inverse-demand Diels-Alder reaction; alkene and tetrazole photoclick reaction, and the like.

A composition can be formulated to have one or more mechanical properties that are similar to the corresponding mechanical property of a tissue into or onto which the composition is introduced. As an example, a composition can be formulated such that the composition has an equilibrium modulus that is within 50%, within 40%, within 35%, within 30%, within 25%, within 20%, within 15%, within 10%, or even within 5% of the equilibrium modulus of the tissue being into or onto which the composition is introduced. This is not a requirement, however, as a user may in some instances wish to use a composition that has a mechanical property that differs by at least 50% from the corresponding mechanical property of the tissue of interest.

Aspect 2. The composition of Aspect 1, wherein the polymer chains comprise any one or more of hyaluronic acid, chitosan, heparin, alginate, gelatin, fibrin, polyvinyl alcohol, polyethylene glycol, sodium polyacrylate, an acrylate polymer, polyethylene glycol (PEG), and polyvinylpyrrolidone (PVP).

Aspect 3. The composition of Aspect 2, wherein the polymer chains comprise hyaluronic acid.

Aspect 4. The composition of any one of Aspects 1-3, wherein the agent comprises any one or more of a therapeutic, a carrier, and a dye. One can also add an imaging or contrast agent to the hydrogel to visualize the hydrogel during injection or once implanted.

For a system that releases a single agent over time, one can first determine how much agent is needed to deliver to achieve an effective dose. Based on this information, one can tailor the number of functional groups—for example, azide or biotin—in the hydrogel to achieve this.

After the hydrogel precursor solution is made, one can use a dual chambered syringe to mix and inject the hydrogel precursor solution and the agent desired for attachment. This combined solution can be UV crosslinked in situ; for example at the site of placement, which placement can be by injection. Such a placement can take place at the annulus fibrosus of a subject. The disclosed compositions can be injected or otherwise introduced to the annulus fibrosus or other tissue of a subject, where the compositions can fill defects, tears, or other damage.

The release profile of the agent can depend, for example, on factors like the strength of the bonds linking the functional group to the hydrogel and linking the agent to the functional group. The release profile can also depend on the degradation profile of the hydrogel, which can be tailored by changing the hydrogel crosslinker used. For instance, swapping a dithiol crosslinker with an MMP degradable crosslinker can speed up hydrogel degradation and subsequently agent release. If a carrier is being used, it is possible to vary the release kinetics through varying the properties of the carrier. For instance, polymer-based carriers can release their cargo via degradation of the carrier and so an increase in shell thickness of the carrier can prolong the release kinetics of the cargo from the carrier.

One can also use the disclosed technology to form a system that delivers multiple agents with varying release profiles. The specifics can differ based on the application; one can first decide how many agents are desired to deliver, how much agent is needed to be effective, and what release profiles are desired for each agent.

For example, if one desired immediate delivery of an agent, one can incorporate that agent directly into the hydrogel precursor solution (with no secondary attachments happening) and have it be delivered via bulk diffusion. If one wanted to deliver an additional agent at a later point in time, one can incorporate azide functional groups and incorporate that agent (e.g., with DBCO functionalization) into the precursor solution. If one wanted to deliver a third agent that is released at a still further later time, one can incorporate biotin functional groups and incorporate that agent (with streptavidin functionalization). In this way, one has three different agents being delivered on different time scales in one system. Regarding functional groups, one can use azide-DBCO and biotin-streptavidin reactions, although these are illustrative only and are not limiting. Thus, one can formulate a composition that delivers different agents at different points in time.

Aspect 5. The composition of Aspect 4, wherein the therapeutic comprises any one or more of an antibiotic, an anti-inflammatory therapeutic, a matrix remodeling therapeutic, a cell migration therapeutic, an anti-apoptotic therapeutic, a pro-anabolic therapeutic, or a pro-extracellular matrix forming therapeutic.

Aspect 6. The composition of Aspect 4, wherein the carrier comprises any one or more of a micelle and a lipid nanoparticle, a liposome, a gold nanoparticle, or a polymersome.

Aspect 7. The composition of Aspect 5, wherein the carrier comprises a therapeutic disposed therein or thereon.

Aspect 8. The composition of any one of Aspects 1-7, wherein the agent is coupled to a pendant group of a polymer chain by any one or more of (1) a conjugate resulting from reacting an azide and alkyne and (2) a streptavidin-biotin bond.

One can use thiol-modified linkers to enable the azide-alkyne and streptavidin-biotin reactions; if an agent is thiol-modified, the agent can be attached to the polymer chain. If a dithiol linker is used, amine-modified agents can be attached.

Aspect 9. The composition of any one of Aspects 1-8, wherein the hydrogel is characterized as being fibrous in nature.

Aspect 10. The composition of any one of Aspects 1-8, wherein the hydrogel is characterized as being granular in nature.

Aspect 11. The composition of anyone of Aspects 1-8, wherein the hydrogel is characterized as a bulk hydrogel.

Aspect 12. A method, comprising curing a precursor composition so as to give rise to a composition according to any one of Aspects 1-8.

Aspect 13. The method of Aspect 12, further comprising introducing the precursor composition to a subject.

Aspect 14. The method of Aspect 13, wherein the introducing comprises injecting. Introducing can also be accomplished by implanting, for example by use of surgical instruments. The precursor composition can be crosslinked and/or functionalized in situ, as described elsewhere herein. Without being bound to any particular theory or embodiment, injecting the precursor composition can give rise to improved penetration and spread of the precursor material into the desired location or locations.

Aspect 15. A precursor composition, comprising: polymer chains bearing pendant groups; a crosslinker adapted to couple pendant groups to one another; and a functionalizer adapted to couple to a pendant group.

Aspect 16. The precursor composition of Aspect 15, wherein the polymer chains comprise hyaluronic acid.

Aspect 17. The precursor composition of any one of Aspects 15-16, wherein the pendant group comprises norbornene.

Aspect 18. The precursor composition of any one of Aspects 15-17, wherein the functionalizer comprises any one or more of an azide, biotin, or streptavidin.

As shown in FIG. 7, a crosslinker can be a dithiol, and a functionalizer can comprise a thiol and an azide. It is not a requirement that the crosslinker and the functionalizer have the same end group, for example, a thiol. The selection of end group can depend on what the hydrogel backbone is and what types of reactions the backbone can have with a the crosslinker or agent linker. As an example, in the case of norbornene-modified hyaluronic acid, thiol-ene addition reactions are useful for linking to the norbornene. Further, by having thiol end groups on both the crosslinker and the functionalizer, one can perform functionalization and the crosslinking simultaneously. It should be understood, however, that the crosslinking need not involve the same reaction as the reaction between the functionalizer and the pendant group. Functionalizing and crosslinking can be performed simultaneously, but they can also be performed sequentially. As an example, crosslinking can be effected by a first type of click chemistry reaction, and functionalizing can be effected by a second type of click chemistry reaction.

Aspect 19. The precursor composition of any one of Aspects 15-18, further comprising an agent, the agent comprising a coupler that conjugates with the functionalizer, and the coupler optionally comprising dibenzocyclooctyne (DBCO).

Aspect 20. The precursor composition of Aspect 19, wherein the coupler conjugates with the functionalizer through a click chemistry reaction.

Aspect 21. A method, comprising curing a precursor composition according to any one of Aspects 15-20 so as to give rise to a composition, the composition optionally being a composition according to any one of Aspects 1-11.

Aspect 22. The method of Aspect 21, wherein the curing is performed in situ within a subject or on a subject. This can take place, for example, at a site of injury or a site of surgical repair.

Aspect 23. A method, comprising: forming a hydrogel; and coupling the hydrogel to an agent, the coupling being effected by coupling (1) a pendant group borne by a polymer chain of the hydrogel and (2) a coupler associated with the agent.

Aspect 24. The method of Aspect 23 wherein the forming the hydrogel comprises reacting polymer chains bearing pendant groups and a crosslinker adapted to couple pendant groups to one another.

Aspect 25. The method of Aspect 23, wherein the coupling comprises a click chemistry reaction.

Aspect 26. The method of Aspect 25, wherein the click chemistry reaction comprises any one or more of an azide-alkyne reaction and a biotin-streptavidin reaction.

Aspect 27. The method of any one of Aspects 23-26, wherein the hydrogel comprises hyaluronic acid.

Aspect 28. The method of Aspect 27, wherein the pendant group comprises norbornene.

Aspect 29. The method of Aspects 27, wherein the pendant group is functionalized.

Aspect 30. The method of any one of Aspects 23-29, wherein the agent comprises any one or more of a therapeutic, a carrier, and a dye.

What is claimed:

1. A composition, comprising:
a hydrogel comprising crosslinked polymer chains,
at least some of the crosslinked polymer chains having an agent coupled thereto.

2. The composition of claim 1, wherein the polymer chains comprise any one or more of hyaluronic acid, chitosan, heparin, alginate, gelatin, fibrin, polyvinyl alcohol, polyethylene glycol, sodium polyacrylate, an acrylate polymer, polyethylene glycol (PEG), and polyvinylpyrrolidone (PVP).

3. The composition of claim 2, wherein the polymer chains comprise hyaluronic acid.

4. The composition of claim 1, wherein the agent comprises any one or more of a therapeutic, a carrier, and a dye.

5. The composition of claim 4, wherein the therapeutic comprises any one or more of an antibiotic, an anti-inflammatory therapeutic, a matrix remodeling therapeutic, a cell migration therapeutic, an anti-apoptotic therapeutic, a pro-anabolic therapeutic, or a pro-extracellular matrix forming therapeutic.

6. The composition of claim 4, wherein the carrier comprises any one or more of a micelle and a lipid nanoparticle, a liposome, a gold nanoparticle, or a polymersome.

7. The composition of claim 5, wherein the carrier comprises a therapeutic disposed therein or thereon.

8. The composition of claim 1, wherein the agent is coupled to a pendant group of a polymer chain by any one or more of (1) a conjugate resulting from reacting an azide and alkyne and (2) a streptavidin-biotin bond.

9. The composition of claim 1, wherein the hydrogel is characterized as being fibrous in nature.

10. The composition of claim 1, wherein the hydrogel is characterized as being granular in nature.

11. The composition of claim 1, wherein the hydrogel is characterized as a bulk hydrogel.

12. A method, comprising curing a precursor composition so as to give rise to a composition according to claim 1.

13. The method of claim 12, further comprising introducing the precursor composition to a subject.

14. The method of claim 13, wherein the introducing comprises injecting.

15. A precursor composition, comprising:
polymer chains bearing pendant groups;
a crosslinker adapted to couple pendant groups to one another; and
a functionalizer adapted to couple to a pendant group.

16. The precursor composition of claim 15, wherein the polymer chains comprise hyaluronic acid.

17. The precursor composition of claim 15, wherein the pendant group comprises norbornene.

18. The precursor composition of claim 15, wherein the functionalizer comprises any one or more of an azide, biotin, or streptavidin.

19. The precursor composition of claim 15, further comprising an agent, the agent comprising a coupler that conjugates with the functionalizer, and the coupler optionally comprising dibenzocyclooctyne (DBCO).

20. The precursor composition of claim 19, wherein the coupler conjugates with the functionalizer through a click chemistry reaction.

21. A method, comprising curing a precursor composition according to claim 15 so as to give rise to a composition.

22. The method of claim 21, wherein the curing is performed in situ within a subject or on a subject.

23. A method, comprising:
forming a hydrogel; and
coupling the hydrogel to an agent,
the coupling being effected by coupling (1) a pendant group borne by a polymer chain of the hydrogel and (2) a coupler associated with the agent.

24. The method of claim 23 wherein the forming the hydrogel comprises reacting polymer chains bearing pendant groups and a crosslinker adapted to couple pendant groups to one another.

25. The method of claim 23, wherein the coupling comprises a click chemistry reaction.

26. The method of claim 25, wherein the click chemistry reaction comprises any one or more of an azide-alkyne reaction and a biotin-streptavidin reaction.

27. The method of claim 23, wherein the hydrogel comprises hyaluronic acid.

28. The method of claim 27, wherein the pendant group comprises norbornene.

29. The method of claim 27, wherein the pendant group is functionalized.

30. The method of claim 23, wherein the agent comprises any one or more of a therapeutic, a carrier, and a dye.
